(19) United States
(12) Patent Application Publication
(10) Pub. No.: US 2025/0325353 A1
(43) Pub. Date: Oct. 23, 2025

(54) 3D PRINTING MOUTH PIECE INCLUDING SIMULATION OF DEFORMATION

(71) Applicant: LAKE3D HOLDING B.V., BB Grubbenvorst (NL)

(72) Inventors: Jessica Pepe, AE Eindhoven (NL); Rachelle Groot, AE Eindhoven (NL); Kim Maria Johanna Alards, AE Eindhoven (NL); Felicia Georgia Ionascu, AE Eindhoven (NL); Joost Anne Veerman, AE Eindhoven (NL)

(21) Appl. No.: 18/874,651

(22) PCT Filed: Jun. 16, 2023
(86) PCT No.: PCT/EP2023/066275

§ 371 Date: Dec. 13, 2024

(30) Foreign Application Priority Data
Jun. 17, 2022 (NL) 2032211

(51) Int. Cl. A61C 13/00; A61C 7/00; A61C 7/08; B33Y 10/00; B33Y 30/00; B33Y 50/02; B33Y 80/00

(52) U.S. Cl. CPC A61C 13/0004; A61C 7/002; A61C 7/08; A61C 13/0019; B33Y 10/00; B33Y 30/00; B33Y 50/02; B33Y 80/00

(57) ABSTRACT

A method for manufacturing a mouth piece, in particular by jetting droplets of a first droplet material and a second droplet material in a 3D printing process. The method comprises a step of obtaining dental data of a user and an input mouth piece model. A simulation is carried out to determine a deformation of the input mouth piece model in response to an exerted force. Based on the deformation, the input mouth piece model is adapted to a work mouth piece model by substituting material of at least a portion of the input mouthpiece model by a replacement material. Finally, the mouth piece is manufactured based on the optimised work mouth piece model.

The present invention relates to a method for manufacturing a mouth piece. In particular, a method for manufacturing by additive deposition of individual droplets from jetting heads, more in particular by jetting droplets of a first droplet material and a second droplet material. The method comprises the steps of:
obtaining dental data of a user including a 3D teeth model of a virtual upper jaw and/or a virtual lower jaw resembling the upper jaw and/or the lower jaw of the users mouth;
obtaining an input mouth piece model;
carrying out a simulation in which the input mouth piece model is positioned on at least one of the virtual upper jaw and virtual lower jaw;
adapting the input mouth piece model to a work mouth piece model;
manufacturing the mouth piece based on the work mouth piece model.

U.S. Pat. No. 10,188,485B2 discloses a dental appliance which can protect the teeth from clenching and bruxism. The dental appliance comprises a cover frame in which a teeth recess is formed in a length direction. The cover frame consists of a laminar structure comprising a first cover layer of a hard material and a second cover layer of soft material provided on the inside relative to the first cover layer. A core frame of a hard material is provided in the teeth recess and has a tooth-contacting surface shaped with impressions of end portions of a plurality of the teeth.

The dental appliance is configured to be excellent in durability and wearing comfort. It can protect the teeth from a heavy occlusal force when clenching or grinding. The dental appliance include a mouthpiece, which may be mounted on the teeth to protect the teeth from external shock applied to a face by a punch or a ball, etc. during workouts such as martial arts or ball games. The mouthpiece may be customised for individuals.

WO2012/140021 discloses a method for manufacturing an orthodontal appliance in a customised way including a step of a simulation.

The orthodontal appliance may have a plurality of embodiments. In some embodiments the orthodontic appliance is selected from the group of braces, brackets, splints, retainers, arch-wires, aligners and shells. In some embodiments, a virtual orthodontic appliance is configured to provide that a manufactured orthodontic appliance hinders the patients from grinding his teeth. In some embodiments, a virtual orthodontic appliance is configured to provide that a manufactured orthodontic appliance is to be comfortable to wear for the patient. In some embodiments, the effect of the orthodontic appliances is to provide protection of the set of teeth, such as where the orthodontic appliance comprises a mouthguard. The orthodontic appliance may comprise a teeth protection device.

In the method, a virtual orthodontic appliance is generated which comprises a first part configured for being positioned at a first section of a virtual 3D teeth model of the patient's teeth. The virtual 3D teeth model comprises a virtual upper jaw and a virtual lower jaw resembling the upper jaw and lower jaw, respectively, of the patient's mouth. In the method, an initial shape of the virtual orthodontic appliance is provided. The initial virtual orthodontic appliance may be provided by selecting among predefined virtual orthodontic appliances from a library. A target virtual dynamical articulation for the set of teeth is determined. A virtual dynamical articulation is performed with the virtual orthodontic appliance positioned anatomically correct at the 3D model, and the virtual orthodontic appliance is adjusted based on a result of the virtual dynamical articulation.

In some embodiments, the method comprises defining a target contact distribution between a part of the virtual orthodontic appliance and a section of the virtual 3D teeth model. When the orthodontic appliance manufactured from the virtual orthodontic appliance is arranged at the patient's teeth, the portions of the orthodontic appliance corresponding to the target contact distribution contacts the patient's teeth. The effect threshold value may relate to a measure of the contact distribution over one or more surfaces of the teeth, such as the occlusal surfaces of the teeth, during occlusion if the orthodontic appliances manufactured from the present form of the virtual model. The effect threshold value may comprise a two-dimensional mapping of the contact distribution over the occlusal surfaces of all these in the first section of the virtual 3D teeth model or over selected teeth. The virtual orthodontic appliance may be adjusted if a result of e.g. a virtual dynamical articulation shows that the present contact distribution differs from the target contact distribution by more than a contact threshold value.

In some embodiments, the effect of the orthodontic appliance on the patient is estimated from the distribution of collision points measured using the virtual dynamic articulation. The collision points may e.g. appear at collisions between the parts of the virtual orthodontic appliance and a section of the virtual 3D teeth model. In some embodiments, the method comprises adjusting the virtual orthodontic appliance based on the estimated effect of the orthodontic appliance.

The orthodontic appliance may be manufactured from the virtual orthodontic appliance using different techniques. The techniques may comprise wax and casting, 3D printing, milling, shaping metal part such as cables and plates. The techniques may be performed alone or in combination. The manufacturing of the orthodontic appliance may comprise a two-material process where different portions of the orthodontic appliance are manufactured in different materials.

In some embodiments, the properties of the material(s) used for manufacturing the orthodontic appliance are taken into account when generating the virtual orthodontic appliance. The material properties may be included in the generation of the virtual orthodontic appliance. Using a flexible material at a tooth contact surface of the manufactured orthodontic appliance may allow for some undercut at the lower part of the teeth. This may allow for instance retainers to be more securely fixed to the patients teeth.

In some embodiments, the virtual orthodontic appliance is adjusted by an additive process or a subtractive process where material virtually is added or removed from the virtual orthodontic appliance, such as virtually added to a modified surface or virtually removed from the modified surface of the first and/or second part of the virtual orthodontic appliance.

A drawback of this method for manufacturing an orthodontic appliance is that a final product of the manufactured mouthpiece may have an inferior quality. In particular, it is desired to obtain a proper method for adapting an input mouthpiece model to a work mouthpiece model to improve a quality of the manufactured mouthpiece.

Regarding the above-mentioned prior art, it is remarked that any discussion of documents, acts, materials, devices, articles or the like included in the present specification is for the purpose of providing a context for the present invention, and is not to be taken as an admission that any such matters form part of the prior art or were before the priority date of each claim of this application common general knowledge in the field relevant to the present invention.

The general object of the present invention is to at least partially eliminate the above mentioned drawback and/or to provide a usable alternative. More specific, it is an object of the invention to provide a method for manufacturing a mouthpiece based on an improved work mouthpiece model.

According to the invention, this object is achieved by a method for manufacturing a mouthpiece according to claim 1. In particular, the method is configured to manufacture the mouthpiece by 3D printing. More in particular, the mouthpiece is manufactured by an additive deposition of individual droplets from jetting heads, more in particular by jetting droplets of at least a first droplet material and a second droplet material which differs from each other and which allows a manufacturing of the mouthpiece out of a combination of a soft and hard material.

In a step of the method, dental data of the user is obtained. The dental data allows a manufacturing of a customised mouthpiece. The dental data includes a 3D teeth model of a virtual upper jaw and/or a virtual lower jaw resembling the upper jaw and/or the lower jaw of the user's mouth. The dental data may be obtained in several ways, e.g. by scanning an oral cavity with a scanning device. An intraoral scanner to obtain oral information of an individual is widely known.

In a step of the method, an input mouthpiece model is obtained. An input mouthpiece model may be obtained from a library file which defines a preset shape of the mouthpiece to be manufactured. The input mouthpiece model may represent any kind of mouthpiece, e.g. an occlusal splint or a mandibular advancement device which includes an upper and lower splint. The mouthpiece to be manufactured may be configured to cover mandibular or maxillary teeth or only a portion of a user's teeth. A mouthpiece may be provided for any kind of purposes, e.g. for a reposition of teeth, to treat bruxism, as a mouthguard to protect during sport activities etc.

In a step of the method, a simulation is carried out in which the input mouthpiece model is positioned on at least one of the virtual upper jaw and/or lower jaw. In the simulation, a deformation of the input mouthpiece model in response to an exerted force is determined. The exerted force may be a bite force or an external impact force, e.g. a force generated by bruxism or as occurring in martial sports.

In particular, in the method, a bite force is simulated to obtain the deformation of the input mouthpiece model. A bite force input obtained in the method may be a standardised bite force being acknowledged in literature for a particular group of users or a particular treatment. Preferably, a bite force input in the simulation is individualised for a particular user. The bite force input may contain measured data and uploaded as a bite force input to carry out the simulation for an individual user.

The deformation of the input mouthpiece model provides information on occurring stresses at certain locations of the mouthpiece. This deformation provides information on how the input mouthpiece model should be adapted to a work mouthpiece model which will form a basis for 3D printing the mouthpiece. Preferably, the simulated deformation of the mouthpiece is presented by a colour map to an operator. Different colours in the map visualise where a possible adaption of the input mouthpiece model may be desired. The operator may inspect the presented deformation and decide on how to adapt the input mouthpiece model to obtain a proper work mouthpiece model. In an embodiment, the method may comprise control electronics which are programmed to adapt the input mouthpiece model based on a determined deformation. Based on software rules, the input mouthpiece model may be adapted and possibly re-simulated to obtain a work mouthpiece model providing an admissible deformation when the mouthpiece is subjected to the exerted force.

In a step of the method, the input mouthpiece model is adapted to the work mouthpiece model by substituting material of at least a portion of the input mouthpiece model by a replacement material. After carrying out at least one adaption, based on the work mouthpiece model, the mouthpiece can be manufactured, in particular by 3D printing.

The method according to the invention is beneficial in that the simulation of a deformation of the mouthpiece as a result of exerted forces in use may timely reveal an undesired deformation of the mouthpiece. A small local plastic deformation may be such an undesired deformation. An undesired deformation may affect a wearing comfort of the mouthpiece. More severe, a simulated mouthpiece deformation may be so large that the mouthpiece loses its functionality in use. In an extreme situation, a deformation might even cause a local crack in the mouthpiece.

By carrying out the simulation, an undesired deformation is detected in an early stage before actually manufacturing the mouthpiece. In case that the simulation reveals an unacceptable deformation, the input mouthpiece model will be adapted by locally substituting material by a replacement material to reduce or increase an occurring deformation. The replacement material replaces a previous defined material in the input mouthpiece model which locally hardens or softens the mouthpiece to alter an occurring deformation to acceptable proportions.

Preferably, the step of simulating a deformation and adaptation of the input mouthpiece model is iteratively carried out until a final work mouthpiece model is obtained to serve as a basis for 3D printing the mouthpiece. In the preferred iterative process, the simulation is carried out multiple times on a modified input mouth piece model including the replacement material to evaluate a modified deformation.

Preferably, the input mouthpiece model is initially configured out of a single material. Before carrying out a first simulation, the input mouthpiece model, a base-line input mouthpiece model, contains a single material, in particular a soft print material. The simulation commences with a one-material input mouthpiece model. Preferably, the single material of the input mouthpiece model is softer than the replacement material. After a step of simulation and adaptation, the input mouthpiece model is adapted and locally contains at least one portion of replacement material.

In an embodiment of the method according to the invention, the replacement material has a composition containing only hard print material. The hard print material may be configured to substitute material portions of the input mouthpiece model being subjected to a maximal deformation. The operator may appoint areas or volumes of material to be replaced with replacement material. At a portion of maximal deformation, a highest stress will occur. By the substitution with hard material, an amount of deformation will be reduced. Herewith, a fit and functionality of the mouthpiece to an upper or lower jaw may be improved. An improved teeth protection may be obtained and bruxism may be prevented.

A 3D printing soft or hard material is commonly defined by a predetermined flexural strength. Typically the soft print material has a flexural strength in a range of at least 1.0 MPa and at most 10 MPa, in particular about substantially 2.0 MPa. Typically, the hard material has a flexural strength in a range of at least 60 MPa, in particular at least 80 MPa.

In an embodiment of the method according to the invention, the replacement material may be a mixture of hard print material and soft print material. The replacement material may be a mixture of a plurality of print materials having at least an amount of a hard print material and an amount of soft print material. The replacement material has a certain mixing ratio of the hard and soft print material. The mixture is suitable for substituting a portion of material being subjected to an intermediate value of stress. The intermediate value of stress is in between the highest and lowest stress corresponding with the simulated maximal and minimal deformation.

In an embodiment of the method according to the invention, a mixing ratio of the mixture of hard and soft print material to anticipate on a simulated deformation may be determined by an empirical process. For a printing process, a mixing ratio may be established to be applied in case a certain deformation is determined in the step of simulation. Herewith, the mixing ratio of the mixture may be defined in a manner to adequately anticipate on occurring deformations.

In an embodiment of the method according to the invention, the mixing ratio of the mixture of hard and soft print material is taken proportional with a certain deformation. In the method, a linear dependency is assumed between a simulated deformation and the mixing ratio of replacement material for adaptation. Preferably, a volumetric ratio of the mixture is linearly related to a degree of deformation. For adaptation, the mixture may contain no hard material for a portion of minimal deformation and no soft material for a portion of a maximal deformation.

The mixture contains a proportional amount of hard and soft print material for a deformation being in between the minimal and maximal deformation. Preferably, the mouthpiece is manufactured by a 3D printer including jetting heads for jetting droplets of a first droplet material and a second droplet material. Preferably, one of the first and second droplet material define the hard material, and the respective other of the first and second droplet material define the soft material.

In an embodiment of the method according to the invention, a simulated deformation is subdivided in at least three ranges, for example including a range of a high, low and intermediate deformation.

Preferably, the input mouthpiece model is meshed. The meshed input mouth piece model may be subdivided into slices, wherein each slice includes a matrix of pixels.

In an embodiment of the method according to the invention, the method has a step of appointing a replacement material for a surface voxel and appointing a replacement material for an interior voxel. Preferably, in a step of method, a local deformation is first determined at the surface voxel of the input mouthpiece model, in particular at a teeth contact surface, for simulating a deformation at a contact surface before determining a deformation at the interior voxel.

In an embodiment of the method according to the invention, the simulation only provides a deformation at the surface voxel. Based on a surface deformation, a material property of a portion having a certain depth may be defined. In an adaptation software, an algorithm may be defined to define interior material properties based on the surface deformation. At least two input parameters may be used to define the interior material properties in which the at least two input parameters include a penetration depth and a gradient of a transition between materials.

In an embodiment of the method according to the invention, the deformation of the input mouthpiece model is defined by a deformation model. In a step, at least one deformation portion is allocated. A positioning of the deformation portion having a particular deformation, for example a low, high or an intermediate deformation is determined. Preferably, a portion depth is additionally defined for a certain deformation portion to define a volume of the deformation portion to be adapted. The deformation portion is in a step of the method at least partially replaced by a replacement material corresponding with the determined amount of deformation.

Preferably, the input mouthpiece model is subdivided into slices, wherein in particular after simulation a droplet material is defined in the work mouthpiece model in a separate bitmap for a certain slice.

Further, the invention relates to a 3D-printer comprising control electronics which are programmed to carry out the method according to the invention.

Further, the invention relates to a computer program product comprising a computer readable medium, the computer readable medium having computer readable code embodied therein, the computer readable code being configured such that, on execution by a suitable computer or processor, the computer or processor is caused to perform the method according to the invention.

Further, the invention relates to a mouthpiece obtained by the method according to the invention.

In an embodiment of the mouthpiece according to the invention, the mouthpiece has a local patch of replacement material. This local patch is configured to compensate for an inadmissible deformation. The local patch is defined by a volume of at least one voxel. In particular, the local patch has a volume of at most 0.3 cm³, in particular at most 0.1 cm³.

In an embodiment of the mouthpiece according to the invention, the local patch is positioned at a client specific position to obtain a customised mouthpiece. Use of the method according to the invention is in particular beneficial to customise a mouthpiece to user specifics, like a deficient tooth of an individual. Compensating for these user specifics results in a mouthpiece having at least one local patch of replacement material at at least one client specific position.

In an embodiment of the mouthpiece according to the invention, the local patch of replacement material is positioned underneath a surface layer of the mouthpiece. The local patch is then fully embedded in the mouthpiece. The local patch is applied at a certain depth inside the mouthpiece, e.g. at a depth of 3 mm. Herewith, the local patch functions to compensate or otherwise inadmissible deformations.

The invention will be explained in more detail with reference to the appended drawings. The drawings show a practical embodiment according to the invention, which may not be interpreted as limiting the scope of the invention. Specific features may also be considered apart from the shown embodiment and may be taken into account in a broader context as a delimiting feature, not only for the shown embodiment but as a common feature for all embodiments falling within the scope of the appended claims, in which:

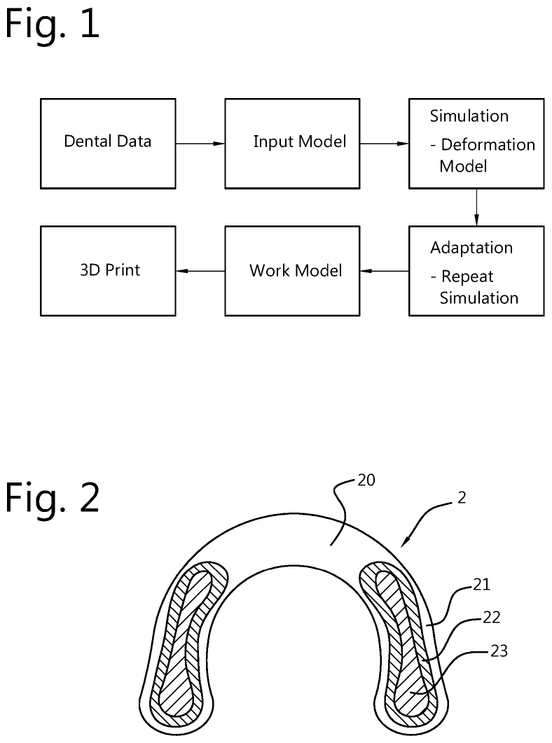

FIG. 1 shows flow diagram of an embodiment of the method according to the invention including a simulation of a deformation of an input mouthpiece model;

FIG. 2 shows a deformation model retrieved by the simulation including areas of different deformations;

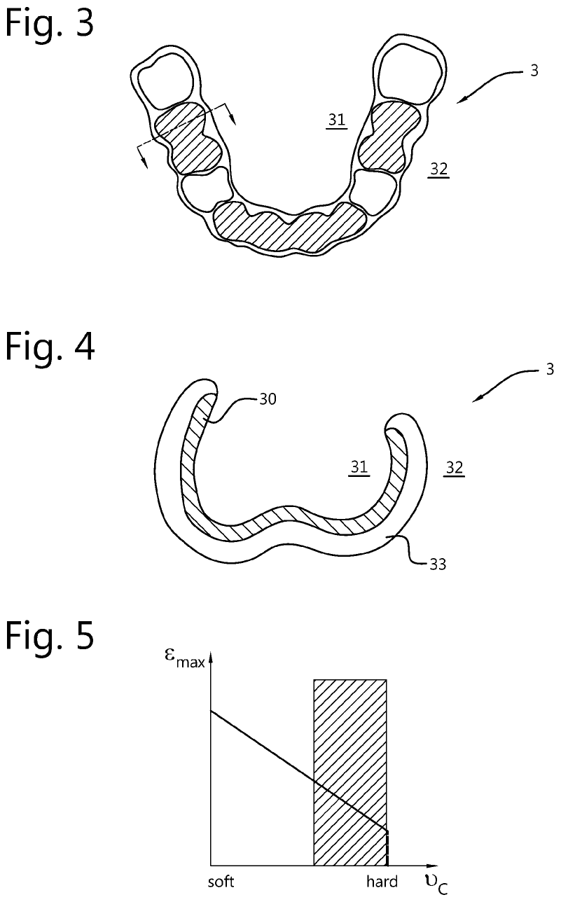

FIGS. 3 and 4 show a work mouthpiece model retrieved after an adaption of the input mouthpiece model serving as a basis for manufacturing the mouthpiece;

FIG. 5 shows a proportional relationship of a mixture of hard and soft replacement material in correspondence with a simulated deformation of the input mouthpiece model.

FIG. 1 shows a graphical representation of an embodiment of the method according to the invention for manufacturing a mouthpiece.

In a first step of the method, dental data is obtained. The dental data of a user includes a 3D teeth model of a virtual upper jaw and/or a virtual lower jaw. The dental data may be acquired by a scanning process by using a scanning tool.

In a step of the method, an input mouthpiece model is obtained. The input mouthpiece model is preferably obtained from a library. Preferably, the input mouthpiece model is defined by a single material.

In a step of the method, a simulation is carried out. In the simulation, the input mouthpiece model is positioned on at least one of the virtual upper jaw and virtual lower jaw. The input mouthpiece model may for example represent a nightguard mouthpiece to be worn on an upper jaw of the user. In the simulation, a virtual articulator can be used to determine a deformation of the input mouthpiece model in response to an exerted force. Preferably, such a simulated exerted force represents a biting force of an individual user. Herewith, a deformation model is obtained.

An example of such a deformation model 2 is illustrated in FIG. 2. The deformation model is presented as a colourmap. Here, different colours are hatched. Each colour represents a certain deformation 21, 22, 23 at a certain location of the input mouthpiece model 20. The colourmap of the deformation model visualises to an operator at which position an adaption of the input mouthpiece model may be required to obtain a proper work mouthpiece model which is usable for manufacturing the mouthpiece. In a more advanced way of carrying out the method, control electronics may be programmed to adapt the input mouthpiece model based on predetermined programmed rules. The control electronics may comprise an algorithm to adapt the input mouthpiece model presenting a particular deformation in response to an exerted force.

Here in FIG. 2, the colourmap of the deformation model 2 indicates portions subjected to a high deformation 23, low deformation 21 and intermediate deformation 22. Initially, the input mouthpiece model may be configured out of a single material. A first obtained deformation model 2 may indicate portions of the input mouthpiece model to be replaced by a replacement material in a first step of adaptation. Preferably, the steps of simulation and adaptation of the input mouthpiece model are iteratively carried out until a final work mouthpiece model is obtained to serve as a basis for manufacturing the mouthpiece.

In a step of the method, a work mouthpiece model 3 is obtained. The work mouthpiece model 3 may be visualised in a simulation software. FIG. 3 and FIG. 4, in which FIG. 4 is a cross sectional view according to the arrows in FIG. 3, show an example of such a work mouthpiece model 3 which is here resembling an occlusal splint. The work mouthpiece model has a tooth contacting surface 31, an inner lateral side 32, an outer lateral side 32 and an occlusion surface 33. The tooth contacting surface refers to a portion of the occlusal splint which in use faces one or more teeth of the user. Here, the tooth contact surface comprises both a soft material and hard material, and the occlusal surface comprises only a hard material.

Portions of the deformation model determined as subjected to an intermediate deformation may be defined in the work mouthpiece model by a material volume providing an intermediate hardness. The mouthpiece is preferably manufactured by a 3D printing, more preferably by an additive deposition of individual droplets from jetting heads. In particular, the method according to the invention is provided for manufacturing a mouthpiece by a 3D printing process in which multi-material droplets or droplets of different materials are jetted from jetting heads.

Preferably, a first droplet material and a second droplet material is jetted in the printing process. A first droplet material and a second droplet material can be combined in the printing process to obtain a material volume corresponding with the material volume in the work mouthpiece model. One of the first and second droplet material is defined as a soft material and the other as a hard material.

Different droplet materials may be specified on different physical properties. The first droplet material may for example be selected on a certain elasticity defined by a Young modulus and the second droplet material on another elasticity defined by another Young modulus. A material volume may contain an amount of droplets including the first and second droplet materials resulting in an intermediate elasticity defined by the included droplets of the first and second material.

As shown in FIG. 5, in the method, a presumption may be incorporated in the method in that a simulated deformation of the input mouthpiece model relates proportional to a certain elasticity of the material volume. The presumption may be incorporated in control electronics of a 3D printer for carrying out the method. A maximal deformation corresponds with a material volume fully out of a soft droplet material, and a minimal deformation corresponds with a material volume fully out of a hard droplet material. An intermediate deformation corresponds to a proportional ratio in the first and second droplet material.

Although the present invention has been described in detail, it will be apparent to those skilled in the art that various changes and modifications can be made without departing from the scope of the invention as hereinafter claimed. It is intended that all such changes and modifications be encompassed within the scope of the present disclosure and claims.

Further, it is remarked that any feature of the method according to the invention which is described in the embodiments and/or mentioned in the dependent claims is in itself considered patentable without any dependency to another presented feature. In particular, any measure presented in a dependent claim is also considered patentable without dependency of the independent claim.

Thus, the invention provides a method and a 3D-printer for carrying out the method to manufacture a mouth piece, in particular by jetting droplets of a first droplet material and a second droplet material in a 3D printing process. The method comprises a step of obtaining dental data of a user and an input mouth piece model. A simulation is carried out to determine a deformation of the input mouth piece model in response to an exerted force. Based on the deformation, the input mouth piece model is adapted to a work mouth piece model by substituting material of at least a portion of the input mouthpiece model by a replacement material. Finally, the mouth piece is manufactured based on the optimized work mouth piece model.

1. Method for manufacturing a mouth piece, in particular by additive deposition of individual droplets from jetting heads, more in particular by jetting droplets of a first droplet material and a second droplet material, comprising the steps of:
obtaining dental data of a user including a 3D teeth model of a virtual upper jaw and/or a virtual lower jaw resembling the upper jaw and/or the lower jaw of the users mouth;
obtaining an input mouth piece model which defines a preset shape of a mouthpiece to be manufactured;
carrying out a simulation in which the input mouth piece model is positioned on at least one of the virtual upper jaw and virtual lower jaw and wherein a deformation of the input mouth piece model is determined in response to an exerted force;
adapting the input mouth piece model to a work mouth piece model by substituting material of at least a portion of the input mouthpiece model by a replacement material;
manufacturing, in particular by 3D printing, the mouth piece based on the work mouth piece model.

2. Method according to claim 1, wherein the step of simulating a deformation and the step of adapting the input mouth piece model is iteratively carried out until a final work mouthpiece model is obtained to serve as a basis for manufacturing the mouthpiece.

3. Method according to claim 1, wherein the input mouth piece model is initially defined by a single material, in particular a material which is softer than the replacement material.

4. Method according to claim 1, wherein the material of the input mouthpiece model is in an initial step before a first simulation defined by a soft print material of a predetermined flexural strength, in particular in a range of at least 1,0Mpa and at most 10Mpa, in particular about substantially 2,0Mpa.

5. Method according to claim 1, wherein a composition of the replacement material only contains hard print material of a predetermined flexural strength, in particular of at least 60 MPa, more in particular 80Mpa.

6. Method according to claim 1, wherein the replacement material is a mixture having at least an amount of a hard print material and an amount a soft print material.

7. Method according to claim 6, wherein an amount of hard material in the mixture of the replacement material is proportional with a certain deformation εₓ between a determined minimal deformation £mm and a determined maximal deformation εₘₐₓ, such that the composition contains no hard material for a portion of a minimal deformation εₘᵢₙ and no soft material for a portion of a maximal deformation εₘₐₓ.

8. Method according to claim 1, wherein a deformation E of the mouth piece is simulated by applying a bite force, wherein in particular the bite force is individualised for a particular user to obtain a customised mouthpiece.

9. Method according to claim 1 comprising a step of appointing a replacement material to a surface voxel and appointing a replacement material to an interior voxel.

10. Method according to claim 9, wherein a replacement material is appointed to a surface voxel before an appointment of a replacement material to the interior voxel.

11. Method according to claim 9, wherein in the step of simulation only a surface deformation is determined, whereafter a replacement material for an interior voxel is determined based on the obtained surface deformation.

12. Method according to claim 1, wherein a simulated deformation of the input mouthpiece model is defined by a deformation model in which at least one deformation portion is allocated.

13. Method according to claim 12, wherein a depth of a deformation portion is determined, wherein in particular the input mouthpiece model is subdivided into slices, wherein in particular after carrying out the simulation, a droplet material is defined in the work mouthpiece model in a separate bitmap for a certain slice.

14. Mouthpiece obtained by a method according to claim 1.

15. Mouthpiece according to claim 14, wherein the mouthpiece has a local patch of replacement material which is configured to compensate for an inadmissible deformation, wherein in particular the local patch has a volume of at most 0.3 cm³, in particular at most 0.1 cm³.

16. Mouthpiece according to claim 15, wherein the local patch of replacement material is positioned at a client specific position to obtain a customised mouthpiece.

17. Mouthpiece according to claim 16, wherein the local patch is positioned at a position of a deficient tooth of a specific client.

18. Mouthpiece according to claim 16, wherein the local patch of replacement material is positioned underneath a surface layer of the mouthpiece.

19. 3D-printer comprising control electronics which are programmed to carry out a method according to claim 1.

20. A computer program product comprising a computer readable medium, the computer readable medium having computer readable code embodied therein, the computer readable code being configured such that, on execution by a suitable computer or processor, the computer or processor is caused to perform the method according to claim 1.
